(19) United States
(12) Patent Application Publication
(10) Pub. No.: US 2026/0209167 A1
(43) Pub. Date: Jul. 23, 2026

(54) PROCESSES AND INTERMEDIATES FOR SYNTHESISING OPICAPONE

(71) Applicant: BIAL - PORTELA & CA, S.A., São Mamede Do Coronado (PT)

(72) Inventors: Joäo Porfirio Da Silva Burgal, Sâo Mamede Do Coronado (PT); Soraia Patricia Pinto Dos Santos, Sâo Mamede Do Coronado (PT); Sarah Filliger, Visp Lonzastrasse (CH); Dominique Roberge, Visp Lonzastrasse (CH); Christof Aellig, Visp Lonzastrasse (CH)

(21) Appl. No.: 19/139,918

(22) PCT Filed: Dec. 22, 2023
(86) PCT No.: PCT/PT2023/050045

§ 371 Date: Jun. 17, 2025

(30) Foreign Application Priority Data
Dec. 23, 2022 (GB) 2219669.5

(51) Int. Cl. C07C 201/08; C07C 205/59; C07D 413/04

(52) U.S. Cl. CPC C07C 201/08; C07C 205/59; C07D 413/04

(57) ABSTRACT

The present invention relates to a method of preparing intermediates for the synthesis of opicapone, which comprises nitration of vanillic acid in an acid anhydride using a nitration agent. The present invention also relates to intermediates for the synthesis of opicapone produced by the process. Furthermore, the invention relates to solutions of intermediates for the synthesis of opicapone in acid anhydride.

### FIELD OF INVENTION

This invention relates to a method of preparing opicapone. In particular, this invention relates to intermediates for preparing opicapone, solutions containing said intermediates and methods of preparing the same. Furthermore, the invention allows intermediates of opicapone to be synthesised in a continuous process in solution using flow chemistry.

### BACKGROUND TO THE INVENTION

Levodopa (L-DOPA) has been used in clinical practice for several decades in the symptomatic treatment of various conditions, including Parkinson's disease. L-DOPA is able to cross the blood-brain barrier, where it is then converted to dopamine and increases the levels thereof. However, conversion of L-DOPA to dopamine may also occur in the peripheral tissue, possibly causing adverse effects upon administration of L-DOPA. Therefore, it has become standard clinical practice to co-administer a peripheral amino acid decarboxylase (AADC) inhibitor, such as carbidopa or benserazide, which prevents conversion to dopamine in peripheral tissue. It is also known that inhibitors of the enzyme catechol-O-methyltransferase (COMT) may provide clinical improvements in patients afflicted with Parkinson's disease undergoing treatment with L-DOPA, since COMT catalyses the degradation of L-DOPA.

It has been found, as set forth in International Publication No. WO 2007/013830, that the nitrocatechol derivative opicapone is a potent and long-acting COMT inhibitor. This compound is bioactive, bioavailable and exhibits low toxicity. Thus, opicapone has potentially valuable pharmaceutical properties in the treatment of some central and peripheral nervous system disorders where inhibition of O-methylation of catecholamines may be of therapeutic benefit, such as, for example, mood disorders; movement disorders, such as Parkinson's disease, parkinsonian disorders and restless legs syndrome; gastrointestinal disturbances; oedema formation states; and hypertension. The development of the opicapone molecule is described in L. E. Kiss et al, J. Med. Chem., 2010, 53, 3396-3411 and it was approved for marketing in the EU in June 2016 as adjunctive therapy to preparations of L-DOPA/AADC in adult patients with Parkinson's disease and end-of-dose motor fluctuations.

WO 2009/116882 describes various polymorphs of opicapone, with polymorph A being both kinetically and thermodynamically stable. WO 2013/089573 describes optimised methods for producing opicapone using simple starting materials and with good yields. The process of WO 2013/089573 employs a cheap and readily available starting material, vanillic acid (Example 1), and the later steps of this process (coupling, oxidation and deprotection steps; Examples 4 to 7) have high yields over 80%. However, the initial nitration step takes place as a slurry in acetic and nitric acids, which must be heated to 90 to 105° C. to form a solution before cooling to recrystallize the crude product. The product is washed with a range of solutions or large volumes of water to recover the final product. The yield of 45 to 55% is moderate to poor. The required additional recrystallization severely impacts efficiency of scale-up.

WO 2007/013830 employs 3,4-dibenzyloxy-5-nitrobenzoic acid as the starting material, thereby avoiding the problems of nitrating vanillic acid. However, the later steps are less efficient with the condensation, dehydration, oxidation and deprotection steps yielding an opicapone-like compound (compound 42, pages 50 to 51) in an overall yield of only 24% (91% then 65% then 54% then 75%).

Therefore, there is a need for an alternative route to synthesise opicapone that can avoid the problems associated with the nitration of vanillic acid described in WO 2013/089573, but that can produce opicapone in a yield greater than in WO 2007/013830. In particular, there is a need for an efficient initial reaction step that is amenable to large-scale production yet allows the later coupling, oxidation and deprotection steps to take place in high overall yield. The process should take place in solution without the need for high temperature (e.g., above 60° C.) and ideally employ a cheap and readily available starting material.

### SUMMARY OF THE INVENTION

The present inventors have now solved this problem by identifying a new process to nitrate vanillic acid in solution (preferably at ambient to moderate temperature (e.g., 15 to 60° C.)). The inventors discovered that by using an acid anhydride (e.g., acetic anhydride) as a solvent, vanillic acid could be dissolved and acylated (preferably at temperatures of 30 to 60° C.) to form an acyl vanillic acid intermediate which could then be reacted with a nitration agent (e.g., nitric acid) to form an acyl nitro-vanillic acid intermediate (preferably at ambient to moderate temperatures (15 to 60° C.)) which, after quenching (e.g., with water), yields the final product nitro-vanillic acid as a crystalline solid without the need to actively lower the temperature or to extensively wash the product. The purity is excellent, thereby avoiding the need to undergo an additional recrystallization step.

Accordingly, in a first general embodiment, the invention provides a method of preparing the compound of formula (VI) (also called nitro-vanillic acid herein):

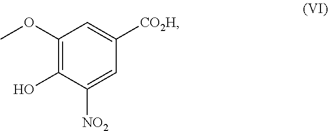

which comprises nitration of vanillic acid in an acid anhydride using a nitration agent.

In a second general embodiment, the invention provides a compound of formula (VI) produced by the process of the first general embodiment.

In a third general embodiment, the invention provides a process for converting the compound of formula (VI) into opicapone.

In a fourth general embodiment, the invention provides a solution of an acyl vanillic acid or an acyl nitro-vanillic acid in an acid anhydride.

### BRIEF DESCRIPTION OF THE FIGURES

FIG. 1 shows a process flow diagram for the nitration of vanillic acid in acetic anhydride by nitric acid to form nitro-vanillic acid in a continuous process using flow chemistry on a laboratory scale.

FIG. 2 shows a process flow diagram for the nitration of vanillic acid in acetic anhydride by acetyl nitrate to form nitro-vanillic acid in a continuous process using flow chemistry on a laboratory scale.

FIG. 3 shows a scaled-up process flow diagram for the nitration of vanillic acid in acetic anhydride by nitric acid to form nitro-vanillic acid in a continuous process using flow chemistry with a continuous stirred tank reactor (CSTR) to quench the reaction products.

FIG. 4 shows a scaled-up process flow diagram for the nitration of vanillic acid in acetic anhydride by nitric acid to form nitro-vanillic acid in a continuous process using flow chemistry with two in-line mixer/reactor flow plates.

### DETAILED DESCRIPTION OF THE INVENTION

### A. Definitions

The following definitions apply to the terms as used throughout this specification, unless otherwise limited in specific instances.

An “acid anhydride” contains the functional group R—(CO)—O—(CO)—R′ and may be formed when one equivalent of water is removed from two equivalents of the organic acids R—COOH and R′—COOH in a dehydration reaction. Generally, R and R′ are each independently an optionally halogenated C₁-C₃ alkyl group. For example, two equivalents of acetic acid (or a halogenated version thereof) may form acetic acid anhydride (or a halogenated version thereof). The terms “acetic acid anhydride”, “acetic anhydride” and “Ac₂O” are equivalent. The skilled person is aware that an acid anhydride may contain a proportion of the organic acid(s) from which it is made.

“Vanillic acid” means 4-hydroxy-3-methoxybenzoic acid.

An “acyl vanillic acid” means a 4-acyloxy-3-methoxybenzoic acid. For example, acetyl vanillic acid means 4-acetyloxy-3-methoxybenzoic acid.

“Nitro-vanillic acid” means 4-hydroxy-5-methoxy-3-nitrobenzoic acid.

An “acyl nitro-vanillic acid” means a 4-acyloxy-5-methoxy-3-nitrobenzoic acid. For example, acetyl nitro-vanillic acid means 4-acetyloxy-5-methoxy-3-nitrobenzoic acid.

An “acyl” functional group means R—(CO)—. Generally, R is an optionally halogenated C₁-C₃ alkyl group.

An “acyloxy” functional group means R—(CO)—O—. Generally, R is an optionally halogenated C₁-C₃ alkyl group.

A “C₁-C₃ alkyl” means a monovalent unsubstituted saturated straight-chain or branched-chain hydrocarbon radical having from 1 to 3 carbon atoms. These can be methyl, ethyl, n-propyl or isopropyl. In the case of the acid anhydrides and acyloxy functional groups disclosed herein, methyl (or the halogenated versions thereof) is preferred for reasons of cost and compatibility with the reagents. Acetic anhydride is the most preferred acid anhydride and acetoxy (also called acetyloxy) is the most preferred acyloxy functional group.

A “nitration agent” is a chemical compound known in the art to introduce a nitro group into an organic compound. A common nitration agent is nitric acid (HNO₃), optionally in combination with catalytic amounts of sulphuric acid (H₂SO₄) (e.g., 0.1 to 1%).

A “quenching” step describes the introduction of a material that reacts with any unused reactants and effectively stops a reaction, for example, the addition of water to hydrolyse an acid anhydride to the free acid(s).

A “continuous process” also known as “flow chemistry” or a “flow chemistry process” is a chemical reaction run in a continuously flowing stream rather than by way of standard batch production. In a continuous process, pumps may move fluids containing reactants, reagents, and/or solvents through a system in which the fluids contact one another where there are tubes joining (mixer, tee-connector for example). This allows chemical or physical reactions to occur on mixing and whilst the reagents continue to pass through the system. Whilst a continuous process is amenable to large-scale manufacturing processes, it may be performed at laboratory-scale (e.g., up to 10 g), pilot-scale (e.g., up to 1 kg) and industrial-scale (e.g., more than 1 kg).

A “process flow diagram” or “PFD” is a schematic diagram commonly used in chemical and process engineering to indicate the general arrangement of a flow chemistry system.

An “initial acyl vanillic acid solution” and an “initial nitration solution” are solutions of the two primary reagents prior to mixing. Each solution may be made in one or more steps. For example, an initial acyl vanillic acid solution (e.g., initial acetyl vanillic acid solution) can be made by dissolving and acylating vanillic acid in an acid anhydride (e.g., acetic anhydride). For example, an initial nitration solution can be made by diluting a nitration agent (e.g., nitric acid) in a solvent (e.g., water). Even if this solution is further mixed with other solvents (e.g., sulphuric acid and/or acetic anhydride) it remains an initial nitration solution until mixed with an initial acyl vanillic acid solution to form a “reaction mixture”.

A “mixer” is a vessel, tank, flow reactor (e.g., capillary reactor or tube reactor) or junction where the reaction solutions are mixed actively or passively. On a laboratory scale a Vapourtec microchip may be used. On a larger scale, one or more mixing plates can be used. For example, one or more A5-sized LL-rhombus FlowPlate® may be employed (see A. Macchi et al, Can. J. Chem. Eng., 2019, 97, 2578-2587). The mixer may be temperature-controlled or contain sensors to monitor the progress of the reaction. The reaction will start upon mixing and may be carried out within the mixer or transferred to a separate reactor for the reaction to continue. Where the reaction occurs in the mixer without transfer to a separate reactor, the mixer may also be described as a “mixer/reactor”.

A “reactor” is a vessel, tank, flow reactor (e.g., capillary reactor or tube reactor) or junction where a chemical reaction primarily takes place. The reactor may include active mixing (beyond that achieved by the fluid dynamics of the liquids entering and exiting the tank). Such a reactor may also be described as a “mixer/reactor”. An example is a “continuous stirred tank reactor”. The reaction products can also be collected in the reactor, in which case it may be known as the “reactor/collector”. Quenching may take place in the reactor or in a separate tank/vessel if the quenching results in the rapid precipitation of the desired product.

A “collector” is a vessel, tank, flow cell or junction in which the reaction products are collected.

A “precipitator” is a vessel, tank, flow cell or junction where precipitation of a reaction product primarily takes place (after quenching). If the precipitation is rapid, it may be initiated in the precipitator. If the precipitation is delayed, it may be initiated in the collector and transferred to the precipitator to control precipitation, in particular to improve control of crystallisation. Depending on the product, it may be precipitated and/or collected as an amorphous solid or a crystalline solid.

A “solution” is a homogeneous liquid mixture in which the minor component (the solute) is uniformly distributed within the major component (the solvent). It contains substantially no solute in solid form.

A “solution . . . in an acid anhydride” or a compound “dissolved in an acid anhydride” means that the acid anhydride is the primary solvent in which the compound (e.g., acyl vanillic acid or acyl nitro-vanillic acid) is dissolved. It encompasses a situation where vanillic acid is initially dissolved and acylated in an acid anhydride then mixed with another solvent (e.g., water) so long as the acyl vanillic acid remains in solution. Preferably, the acid anhydride accounts for at least 50% w/w of the solvent, so is the primary solvent. The term “acyl vanillic acid in an acid anhydride” means the acid anhydride is present in quantities sufficient to solubilise the acyl vanillic acid (e.g., acetyl vanillic acid) and is present in at least a 10 molar excess compared to the acyl vanillic acid (e.g., acetyl vanillic acid). An acetyl vanillic acid solution in acetic anhydride is preferred.

A “slurry” or “suspension” is a heterogeneous mixture of solids suspended in a liquid.

The concentration of a solute in a solvent is defined as “percentage weight/weight” or “% w/w”. This equates to the number of grams of solute per 100 grams of solution. For example, an initial acyl vanillic acid solution in acetic anhydride containing 10 g of acyl vanillic acid in 100 g of solution, equates to 10% w/w. As a further example, an initial nitric acid solution in water containing 65 g of nitric acid in 100 g of solution, equates to 65% w/w.

In the claims, the word “comprising” does not exclude other elements or steps, and the indefinite article “a” or “an” does not exclude a plurality.

### B. Methods of Synthesising Nitro-Vanillic Acid

In the first general embodiment, the invention provides a method of preparing the compound of formula (VI):

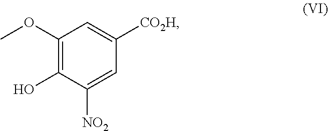

which comprises nitration of vanillic acid in an acid anhydride using a nitration agent.

The inventors surprisingly discovered that use of an acid anhydride solvent system allowed the cheap and readily available vanillic acid starting material to be dissolved and acylated (preferably at moderate temperature (e.g., 30 to 60° C., preferably 35 to 50° C.)) then nitrated (preferably at ambient to moderate temperatures (e.g., 15 to 60° C., preferably 20 to 45° C., more preferably 25 to 40° C.)). Furthermore, carrying out the nitration reaction in this way allowed the product nitro-vanillic acid (compound of formula (VI)) to be recovered upon quenching with low amounts of the side product 6-methoxy-2,4-dinitrophenol, without the need for an additional recrystallization step, in high yield and high purity. Furthermore, the discovery that the nitration reaction could take place in solution at ambient to moderate temperatures without the need for active cooling makes it amenable to synthesis in a continuous process using flow chemistry and allows continuous monitoring of the nitration reaction in small aliquots of the reaction liquid (e.g., during an in-process control).

### Vanillic Acid/Acid Anhydride

The vanillic acid, acid anhydride and nitration agent can be mixed in any order. However, in a preferred embodiment, the vanillic acid is dissolved and acylated in the acid anhydride to form an initial acyl vanillic acid solution (prior to mixing with the nitration agent). This allows the vanillic acid to be fully dissolved and acylated (preferably at moderate temperature (e.g., 30 to 60° C., preferably 35 to 50° C.)) before reaction. If required, the initial acyl vanillic acid solution can be prepared in advance (up to 24 hours before reaction) then stored at low temperatures (0 to 10° C., preferably 0° C.) to prevent side reactions from taking place. The reaction with the nitration agent can then take place (preferably at ambient to moderate temperatures (e.g., 15 to 60° C., preferably 20 to 45° C., more preferably 25 to 40° C.)).

The initial acyl vanillic acid solution can be concentrated or dilute. However, concentrated solutions are generally preferred on larger scales and for storage. The inventors discovered that the solubility of vanillic acid in acid anhydrides (e.g., acetic anhydride) was generally very high. This is believed to result from acylation (e.g., acetylation) of the 4-hydroxy group. In particular, vanillic acid was soluble in acetic anhydride in concentrations up to and including 15% w/w at 45° C. However, at this concentration, precipitation/suspension was observed after addition of a nitration agent (e.g., HNO₃) which makes the process less amenable to operation as a continuous process using flow chemistry. Furthermore, on initiating the reaction at 15% w/w under batch conditions, the inventors detected the formation of a concentrated solution of acetyl nitrate (from the reaction of nitric acid with acetic anhydride), which fumes in moist air and is explosive. Whilst this does not prevent the reaction occurring, it is less preferred for reasons of safety. Therefore, in another preferred embodiment, the initial acetyl vanillic acid solution is prepared by dissolving 3 to 12% w/w vanillic acid in acetic anhydride and comprises 3.75 to 15% w/w acetyl vanillic acid in acetic anhydride. More preferably, the initial acetyl vanillic acid solution is prepared by dissolving 5 to 10% w/w vanillic acid in acetic anhydride and comprises 6.25 to 12.5% w/w acetyl vanillic acid in acetic anhydride. In contrast, the solubility of vanillic acid in acetic acid is only 2.5% w/w under standard conditions.

In order to reduce solvents and optimise the reaction scale, more concentrated initial acyl vanillic acid solutions can be employed. In order to maximise stability, up to 10% w/w vanillic acid may be used to provide initial acetyl vanillic acid solutions up to 12.5% w/w. Therefore, in an even more preferred embodiment, the initial acetyl vanillic acid solution is prepared by dissolving 3 to 10% w/w vanillic acid in acetic anhydride and comprises 3.75 to 12.5% w/w acetyl vanillic acid in acetic anhydride or is even prepared by dissolving 5 to 10% w/w vanillic acid in acetic anhydride and comprises 6.25 to 12.5% w/w acetyl vanillic acid in acetic anhydride.

Generally, the nitration reaction can be run in a variety of simple organic acid anhydrides.

In another preferred embodiment, the reaction takes place in a linear or branched short-chain organic acid anhydride (C₁—(CO)—O—(CO)—C₁, C₂—(CO)—O—(CO)—C₂ or C₃—(CO)—O—(CO)—C₃ wherein C₁, C₂ and C₃ represent methyl, ethyl, and n-propyl or isopropyl respectively (or a halogenated version thereof) with attendant formation of the corresponding acyl vanillic acid. More preferably, the acid anhydride is acetic anhydride (or a halogenated version thereof) with attendant formation of acetyl vanillic acid (or a halogenated version thereof). Even more preferably, the acid anhydride is acetic anhydride or trichloroacetic anhydride with attendant formation of acetyl vanillic acid or trichloroacetyl vanillic acid. Acetic anhydride is most preferred with attendant formation of acetyl vanillic acid.

### Nitration Agent

Preferred embodiments relating to the acid anhydride and vanillic acid can be readily combined with preferred embodiments relating to the nitration agent, described below.

Nitric acid is a liquid, so it can be used without dilution. However, in another preferred embodiment, the nitration agent is provided in the form of an initial nitration solution (prior to mixing with the initial acyl vanillic acid solution). Preferably, the initial nitration solution comprises a solvent selected from the group consisting of water, acetic acid, Ac₂O, or a water/Ac₂O mixture. More preferably the initial nitration solution is an aqueous solution (i.e., water is the primary solvent). These solvents are stable and compatible with the initial acyl vanillic acid solution. The initial nitration solution is amenable to use in a continuous process using flow chemistry. Surprisingly, aqueous solutions of nitric acid (e.g., 50% w/w or greater) can be used even though the water could have been predicted to react with the acetic anhydride and quench the reaction.

The initial nitration solution can be concentrated or dilute. However, concentrated solutions are preferred on larger scales to maximise process efficiency and minimise cost. Therefore, in another preferred embodiment, the initial nitration solution comprises 50 to 99.9% w/w nitration agent. More preferably, the initial nitration solution comprises 65 to 99.9% w/w nitration agent.

In another preferred embodiment, the nitration agent is nitric acid or a derivative thereof, such as acetyl nitrate (which can be formed when nitric acid is in contact with acetic anhydride). More preferably the nitration agent is nitric acid. Nitric acid is a liquid, so can be directly mixed with an initial acyl vanillic acid solution in a pure form. However, in an even more preferred embodiment, the nitric acid is in the form of an initial nitration solution described above in the concentrations described above. Optionally, the initial nitration solution comprises nitric acid (e.g., 50 to 99.9% w/w) with acetic anhydride to form a “mixed acid” (see F. Bordwell and E Garbisch, J. Am. Chem. Soc., 1960, 82, 2578-2587). The use of sulphuric acid can have an accelerating role in the nitration reaction. The use of nitric acid, optionally as a “mixed acid”, may improve the yield.

### Reaction Conditions

In another preferred embodiment, the nitration is carried out at a temperature from about 15 to about 60° C., more preferably at a temperature from about 20 to about 45° C., even more preferably 25 to 40° C. To ensure the temperatures are stable, the vanillic acid and/or acid anhydride and/or nitration agent are preferably pre-heated to the reaction temperature before the nitration reaction.

After the nitration reaction, the reaction may be quenched, preferably wherein the nitration reaction product is directly quenched with water. This not only quenches the nitration reaction, but also converts the available acid anhydride (e.g., acetic anhydride) to the free acid (e.g., acetic acid) in which the compound of formula (VI) has generally much lower solubility. Therefore, the compound of formula (VI) only starts to precipitate once the nitration reaction is quenched, for example, by excess water.

In another preferred embodiment, the quenching is carried out at a temperature from about 4 to about 40° C., preferably from about 10 to about 30° C., more preferably from about 20 to about 25° C. This ensures the optimum amount of the reaction product precipitates in a suitable timeframe.

In another preferred embodiment, a part (or all) of the nitration reaction product is quenched with a 1.5- to 20-fold excess of water. Generally, the nitration reaction is fully quenched on a smaller scale and semi-batch quenched or quenched in a continuous stirred tank reactor (CSTR) when employing a continuous process using flow chemistry. Generally, larger excesses of water (e.g., 5- to 20-fold) are employed in a continuous process using flow chemistry. Larger amounts of water can be used but are unnecessary.

### Continuous Process Using Flow Chemistry

The embodiments relating to acid anhydride, vanillic acid and nitration agent can be readily combined with each other and with preferred embodiments relating to a continuous process using flow chemistry, as described below.

As described above, the use of an acid anhydride solvent system allowed the cheap and readily available vanillic acid starting material to be dissolved and acylated (preferably at moderate temperature (e.g., 30 to 60° C., preferably 35 to 50° C.) then nitrated (preferably at ambient to moderate temperatures (e.g., 15 to 60° C., preferably 20 to 45° C., more preferably 25 to 40° C.)). Furthermore, the reaction allows the product 4-hydroxy-5-methoxy-3 nitrobenzoic acid (compound of formula (VI)) to be recovered with low amounts of the side product 6-methoxy-2,4-dinitrophenol. The discovery that the nitration reaction could take place in solution makes it amenable to synthesis in a continuous process using flow chemistry and allows continuous monitoring of the reaction on small aliquots of the reaction liquid. The fact that the product can be recovered in a high purity without an additional recrystallization step makes it particularly suitable for a continuous process using flow chemistry.

Therefore, in another preferred embodiment, the nitration reaction is carried out as a continuous process in solution. This allows the reaction to be carried out safely, at scale and more efficiently. The prior art process described in WO 2013/089573 is not amenable to synthesis in a continuous process using flow chemistry because the reaction takes place as a slurry or suspension and the product requires additional recrystallization. The prior art process described in WO 2013/089573 discloses yields of ~45%. The use of a continuous process in solution achieved yields of ~70% or even greater.

In another preferred embodiment carried out as a continuous process in solution, the initial acyl vanillic acid solution is prepared by dissolving 3 to 12% w/w vanillic acid in acetic anhydride and comprises 3.75 to 15% w/w acetyl vanillic acid in acetic anhydride. More preferably, the initial vanillic acid solution is prepared by dissolving 5 to 10% w/w vanillic acid in acetic anhydride and comprises 6.25 to 12.5% w/w acetyl vanillic acid in acetic anhydride. In contrast, the solubility of vanillic acid in acetic acid is only 2.5% w/w. The upper limits ensure there is no precipitation when the initial acetyl vanillic acid solution is mixed with the initial nitration solution.

In another preferred embodiment carried out as a continuous process in solution, the initial nitration solution comprises 50 to 99.9% w/w nitration agent. More preferably, the initial nitration solution comprises 65 to 99.9% w/w nitration agent.

When carried out as a continuous process in solution, the initial acyl vanillic acid solution may be mixed with the initial nitration solution in one or more mixers to form a reaction mixture. Preferably, the initial solutions are pre-heated to a temperature from about 15 to about 60° C. This allows the nitration reaction to occur at the preferred temperature from about 15 to about 60° C., more preferably 20 to 45° C., even more preferably 25 to 40° C. The nitration reaction is initiated on contact between the initial acyl vanillic acid solution and initial nitration solution.

When carried out as a continuous process in solution where the initial acyl vanillic acid solution is mixed with the initial nitration solution in one or more mixers (or mixer/reactors) to form a reaction mixture, the initial acyl vanillic acid solution may be introduced into a first mixer (or mixer/reactor) via a first feeding tube preferably at a flow rate 1- to 30-fold higher than the flow rate at which the initial nitration solution is introduced into the first mixer (or mixer/reactor) via a second feeding tube. For example, the initial acyl vanillic acid solution may be introduced into a first mixer (or mixer/reactor) at a flow rate of 10 to 600 g/minute via a first feeding tube. For example, the initial nitration solution may be introduced into a first mixer (or mixer/reactor) at a flow rate of 1 to 60 g/minute via a second feeding tube. Generally, higher flow rates are used on larger scale reactions.

When carried out as a continuous process in solution where the initial acyl vanillic acid solution is mixed with the initial nitration solution in more than one mixer (or mixer/reactors) to form a reaction mixture, the reaction mixture may flow to a second mixer (or mixer/reactor) after mixing in the first mixer (or mixer/reactor). Preferably, the first and/or second mixers (or mixer/reactors) are heated to a temperature from about 15 to about 60° C. This allows the nitration reaction to occur at the preferred temperature from about 15 to about 60° C., more preferably from about 20 to about 45° C., even more preferably 25 to 40° C. The nitration reaction is initiated on mixing by the first mixer (or mixer/reactor), but the yield may be improved by use of a second mixer (or mixer/reactor) because it improves mixing and allows a longer residence time (reaction time).

When carried out as a continuous process in solution where the initial acyl vanillic acid solution is mixed with the initial nitration solution in one or more mixers (or mixer/reactors) to form a reaction mixture, the reaction mixture may subsequently flow to a collector. The nitration reaction may continue in the collector until the nitration reaction is complete or quenched. The nitration reaction is preferably quenched, preferably with water.

When the reaction mixture is quenched with water in the collector it forms a quenched reaction mixture. Preferably, the water is introduced into the collector via a feeding tube. More preferably, the water is introduced into the collector at a flow rate of 50 to 1500 g/minute. Generally, higher flow rates are used on larger scale reactions.

After quenching, the quenched reaction mixture may flow to a precipitator. The compound of formula (VI) may then be collected, preferably as a crystalline solid. Preferably, the compound of formula (VI) is produced from vanillic acid in an overall yield of 50% or greater, more preferably 60% or greater, even more preferably 70% or greater.

The compound of formula (VI) may be precipitated, filtered and subsequently dried. Preferably, the compound of formula (VI) is recovered in a dry crystalline form without an additional recrystallization step.

### C. Downstream Processes to Produce Opicapone

The compound of formula (VI) (i.e., nitro-vanillic acid) is useful to form opicapone.

In the third general embodiment, the compound of formula (VI), produced by the methods in Section B above, may undergo one or more further synthetic steps toward the synthesis of opicapone.

In one further embodiment, a compound of formula (V):

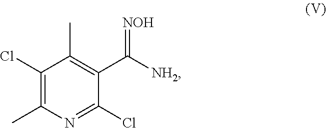

is reacted with the compound of formula (VI) to form a compound of formula (III):

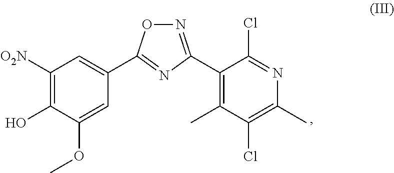 [SMILES: COc1cc(-c2nc(-c3c(Cl)nc(C)c(Cl)c3C)no2)cc([N+](=O)[O-])c1O ; ","]

wherein the compound of formula (VI) is produced by the methods in Section B above. Preferably, the compound of formula (VI) has not undergone an additional recrystallization step. The invention also covers a compound of formula (III) when produced by this process.

In another further embodiment, the compound of formula (III), produced by the methods above, may then be oxidised to form a compound of formula (I):

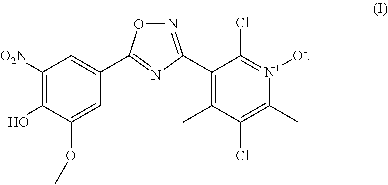

The invention also covers a compound of formula (I) when produced by this process.

In another further embodiment, the compound of formula (I), produced by the methods above, may then be O-demethylated to form a compound of formula (II):

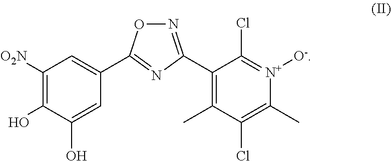

The invention also covers a compound of formula (II) when produced by this process.

### D. Solution of an Acyl Vanillic Acid or an Acyl Nitro-Vanillic Acid in an Acid Anhydride

In the fourth general embodiment, the invention provides a solution of an acyl vanillic acid or an acyl nitro-vanillic acid in an acid anhydride, preferably an acetyl vanillic acid or an acetyl nitro-vanillic acid in acetic anhydride.

The present inventors discovered that concentrated acyl vanillic acid solutions (e.g., acetyl vanillic acid) can be prepared in an acid anhydride (e.g., acetic anhydride), for example at temperatures of about 15 to about 60° C., and reacted with a nitration agent (e.g., nitric acid) to form an acyl nitro-vanillic acid (e.g., acetyl nitro-vanillic acid) which, upon quenching, yields nitro-vanillic acid. In contrast, the solubility of vanillic acid in acetic acid as described in WO 2013/089573 is only 2.5% w/w.

In a preferred embodiment, the solution comprises 4.6 to 18.2% w/w of acetyl nitro-vanillic acid at room temperature and pressure, preferably 7.6 to 15.2% w/w. In another preferred embodiment, the solution comprises 6.4 to 25.6% w/w of trichloroacetyl nitro-vanillic acid at room temperature and pressure, preferably 10.7 to 21.3% w/w.

In another preferred embodiment, the compound of formula (VI) is greater than 50% pure with respect to the total vanillic acid and related organic compounds. More preferably, the compound of formula (VI) is greater than 80% pure, even more preferably greater than 90% pure, most preferably greater than 95% pure, with respect to the total weight of vanillic acid and related organic compounds

In another preferred embodiment, the acid anhydride is a linear or branched short-chain organic acid anhydride (C₁—(CO)—O—(CO)—C₁, C₂—(CO)—O—(CO)—C₂ or C₃—(CO)—O—(CO)—C₃ wherein C₁, C₂ and C₃ represent methyl, ethyl, and n-propyl or isopropyl respectively (or a halogenated version thereof), and the acyl group of the acyl vanillic acid is acetyl, ethanoyl, and n-propanoyl or isopropanoyl respectively (or a halogenated version thereof). More preferably, the acid anhydride is acetic anhydride (or a halogenated version thereof), and the acyl group of the acyl vanillic acid is acetyl (or a halogenated version thereof). Even more preferably, the acid anhydride is acetic anhydride or trichloroacetic anhydride and the acyl group of the acyl vanillic acid is acetyl or trichloroacetyl. Acetic anhydride is most preferred and the acyl group of the acyl vanillic acid is most preferably acetyl.

Based on the discovery of an improved dissolution/acylation and reaction profile for vanillic acid in acid anhydrides, the invention also extends to an acyl vanillic acid solution in an acid anhydride. The vanillic acid was found to dissolve and acetylate in acetic anhydride up to and including 15% w/w. Whilst the resulting 18.75% w/w acetyl vanillic acid solution was not amenable to use in flow chemistry due to precipitation once mixed with nitration agents, it could be utilised on a batch scale. Furthermore, as the acetyl vanillic acid solution can be stored for up to 24 hours before reaction, a 18.75% w/w solution is advantageous for storage or transportation and can be diluted further for use (e.g., at 3.75 to 15% w/w). In particular, the invention provides a 3.75 to 18.75% w/v solution of acetyl vanillic acid in acetic anhydride, preferably 3.75 to 15% w/w acetyl vanillic acid in acetic anhydride, more preferably 6.25 to 12.5% w/w acetyl vanillic acid.

In order to reduce solvents when storing, more concentrated initial acyl vanillic acid solutions can be employed. Up to 18.75% w/w acetyl vanillic acid solution may be used. Therefore, in an even more preferred embodiment, the acetyl vanillic acid solution in acetic anhydride comprises 3.75 to 18.75% w/w acetyl vanillic acid or even 6.25 to 18.75% w/w acetyl vanillic acid.

In a preferred embodiment, the acid anhydride is a linear or branched short-chain organic acid anhydride (C₁—(CO)—O—(CO)—C₁, C₂—(CO)—O—(CO)—C₂ or C₃—(CO)—O—(CO)—C₃ wherein C₁, C₂ and C₃ represent methyl, ethyl, and n-propyl or isopropyl respectively (or a halogenated version thereof), and the acyl group of the acyl vanillic acid is acetyl, ethanoyl, and n-propanoyl or isopropanoyl respectively (or a halogenated version thereof). More preferably, the acid anhydride is acetic anhydride (or a halogenated version thereof), and the acyl group of the acyl vanillic acid is acetyl (or a halogenated version thereof). Even more preferably, the acid anhydride is acetic anhydride or trichloroacetic anhydride and the acyl group of the acyl vanillic acid is acetyl or trichloroacetyl. Acetic anhydride is most preferred and the acyl group of the acyl vanillic acid is most preferably acetyl.

### E. EXAMPLES

### Example 1—Nitration of Vanillic Acid in Acetic Anhydride to Form Nitro-Vanillic Acid (Compound of Formula (VI)) in a Batch Mode

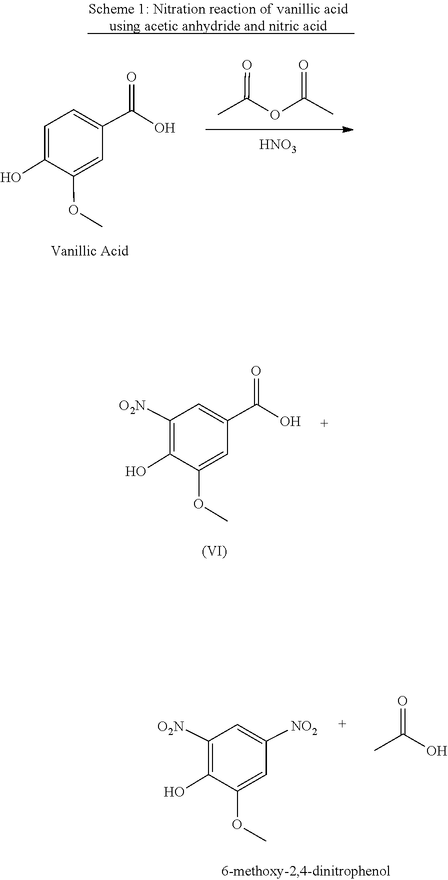

4-hydroxy-3-methoxybenzoic acid (vanillic acid; 12.05 g, 71.7 mmol) was dissolved and acetylated in acetic anhydride (115 mL) in a round bottom flask while heating up to a temperature of 50° C. At lower temperatures dissolution is slower. Once fully dissolved (10% w/w (based on vanillic acid starting material)), the temperature was allowed to reduce to 25° C. and the solution stirred for 0.5 h. The solution was used immediately or stored for up to 24 h at 0° C.

To the solution of acetyl vanillic acid in acetic anhydride at 25° C. was added nitric acid (65% w/w in water, 8.00 mL, 115 mmol) at a flow rate of 0.27 mL/min with a syringe pump over 30 minutes. After the addition, the reaction was left stirring for 1 hour at 25° C. and then quenched with the addition of 200 g water/ice. After 15 minutes, the heavy liquid phase was at the bottom (“oily” phase) and the mixture was left to stir for 1 h to reach room temperature. At this point, precipitation started to occur and the resulting mixture was filtered using vacuum filtration. The precipitate was washed with 200 ml of water and dried in a vacuum oven at 50° C. overnight (m=3.4 g; purity from LC-MS ~95%; yield: 21%). The mother liquor was left overnight, and more precipitate was formed. The precipitate was filtered and left drying overnight in the vacuum oven at 50° C. (m=4.3 g; purity from LC-MS ~82% purity; yield (overall: 23%). It is particularly surprising that the reaction proceeded using nitric acid dissolved in water, which hydrolyses a proportion of the acetic anhydride to acetic acid. Another notable feature is that the intermediates, acetyl vanillic acid and acetyl nitro-vanillic acid, remain in solution throughout the course of the reaction, despite the addition of nitric acid in water. This might have been expected to prevent the reaction from occurring efficiently due to precipitation of the acetyl vanillic acid and/or the acetyl nitro-vanillic acid. Furthermore, after quenching, the final reaction product (nitro-vanillic acid) precipitated reliably over a period of time and could be collected without the need for an additional recrystallization step. Therefore, the reaction described above is amenable to flow chemistry.

### Example 2—Nitration of Vanillic Acid in Acetic Anhydride to Form Nitro-Vanillic Acid (Compound of Formula (VI)) in a Continuous Process Using Flow Chemistry on Laboratory Scale

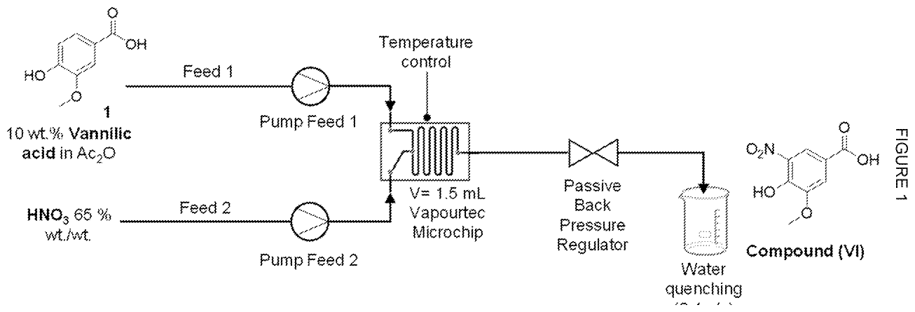

The process of Example 1 was transferred to a continuous process using flow chemistry on a laboratory scale. The process flow diagram is shown in FIG. 1.

Part 2a—Preparation of 10% w/w Acetyl Vanillic Acid Solution in Acetic Anhydride

A solution of 10% w/w acetyl vanillic acid in acetic anhydride (based on vanillic acid starting material) was prepared in a round-bottom flask by first measuring the mass of vanillic acid followed by the addition of acetic anhydride. After this, the solution was heated to 40-50° C. while stirring to achieve full dissolution and acetylation. A clear solution (acetyl vanillic acid 10% w/w (based on vanillic acid starting material)) was obtained. Once fully dissolved, the solution was cooled to room temperature. The solution remained a clear solution, even if placed in the fridge at 4° C. for more than one day. The solution was generally prepared on the day of the experiment to minimise degradation of acetyl vanillic acid and thus avoid the presence of impurities. To confirm the quality of the solution, a 200 μL sample was dissolved in 1 mL of THF:MeCN:H₂O (4:3:3 v/v) and analysed by LC-MS. The purity remained at 99%. Similar solubility and stability were achieved with trichloroacetic anhydride, albeit as a milky, more viscous solution.

Therefore, acyl vanillic acids are highly soluble and stable in acid anhydrides, especially acetyl vanillic acid in acetic anhydride.

Part 2b—Nitration of 10% w/w Acetyl Vanillic Acid Solution in Acetic Anhydride by Nitric Acid

The flow setup consisted of two HPLC pumps (2×Knauer pumps Vapourtec) to introduce a solution of 10% w/w acetyl vanillic acid in acetic anhydride (based on vanillic acid starting material) (feeding tube 1; 0.378-5.149 mL/min) and nitric acid 65% w/w aq. (feeding tube 2; 0.016-0.358 mL/min). The reaction included 1.6 molar equivalents of HNO₃ per mole of vanillic acid starting material. The process flow diagram is shown in FIG. 1. Feeding tubes 1 and 2 were directly pumped using HPLC pumps 1 and 2, respectively. Before the experiment commenced, the reactor setup was flushed by pumping glacial acetic acid through pumps 1 and 2 at a flow rate of 1 mL/min. Subsequently, 10% w/w acetyl vanillic acid in acetic anhydride (based on vanillic acid starting material) (feeding tube 1) and 65% w/w nitric acid (feeding tube 2) were introduced into the flow system at a flow rate dependent on residence time and equivalents of HNO₃ (in this case 1.6 molar equivalents of nitric acid; 40° C.; 5 mL/min 10% w/w acetyl vanillic acid in acetic anhydride (based on vanillic acid starting material); 0.358 mL/min HNO₃). To initiate the reaction, pumps 1 and 2 were switched from glacial acetic acid to feeding tube 1 (10% w/w acetyl vanillic acid in acetic anhydride (based on vanillic acid starting material)) and feeding tube 2 (nitric acid 65% w/w aq.), and these feeds were combined in a Vapourtec mixer/reactor microchip (1.0 mm channel width, residence volume V1=1.5 mL) at 20 or 40° C. As soon as the system reached thermal steady state, the corresponding fraction was collected. On exiting the microchip mixer/reactor, the reaction mixture was diluted to form a reaction mixture:water mixture (2:1 v/v). A sample of reaction mixture was submitted for analysis by HPLC.

The product contained up to 91% nitro-vanillic acid (compound of formula (VI)) and as little as 5.5% of the side product 6-methoxy-2,4-dinitrophenol.

### Example 3—Nitration of Vanillic Acid in Acetyl Nitrate to Form Nitro-Vanillic Acid (Compound of Formula (VI)) in a Continuous Process Using Flow Chemistry on Laboratory Scale

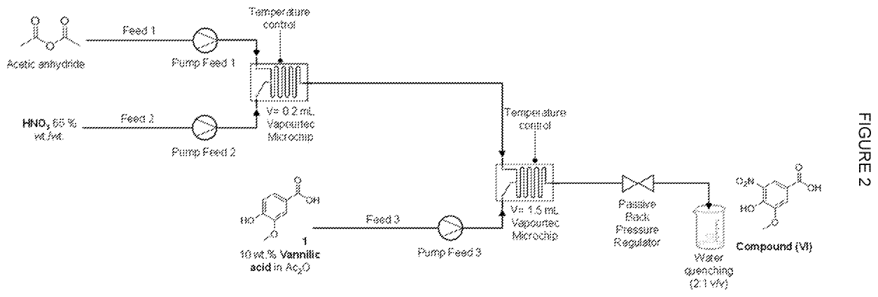

The process of Example 2 was modified so the nitric acid solution was initially mixed with acetic anhydride to form acetyl nitrate on a laboratory scale. The process flow diagram is shown in FIG. 2.

The premixing of the initial nitric acid solution with acetic anhydride prior to mixture with the initial acetyl vanillic acid solution to form the reaction mixture necessitated an additional mixer/reactor and pump. The flow rates and two temperatures (for mixer/reactor 1 and mixer/reactor 2) could be varied independently. Increasing the equivalents of HNO₃ to vanillic acid starting material from 0.68 to 1.6 increased the yield from 46% to 67%. The conditions from the best result obtained (0.28 min residence time, 40° C. and 1.6 eq. of HNO₃ to vanillic acid starting material) were carried forward for further testing. For simplicity, both mixer/reactors were operated at the same temperature (40° C.). The flow setup consisted of 2 HPLC pumps (2×Knauer pumps Vapourtec) and 1 peristaltic pump (V-3) with chemically resistant tubing (compatible with acetic anhydride and acetic acid) to introduce a solution of 10% w/w acetyl vanillic acid in acetic anhydride (based on vanillic acid starting material) (feeding tube 3), nitric acid 65% w/w in water (feeding tube 2) and acetic anhydride (feeding tube 1). Feeding tubes 2 and 1 were directly pumped using HPLC pumps 2 and 1. Before the experiment commenced, the flow system was flushed by pumping glacial acetic acid at a flow rate of 1 mL/min using pumps 1, 2 and 3. Subsequently, nitric acid (feeding tube 2; 0.337 mL/min) and acetic anhydride (feeding tube 1; 0.457 mL/min) were introduced into the flow system at a flow rate to ensure the desired residence time and equivalents of HNO₃ to vanillic acid starting material (1.6). To commence the experiment, pumps 2 and 1 were switched from glacial acetic acid to feeding tube 2 and feeding tube 1 and these feeds were mixed in a Vapourtec microchip (1.0 mm channel width, residence volume V1=0.2 mL) at 40° C. The microchip mixer/reactor was heated using heated air circulating around the microchip (a thermocouple sits directly on the reactor wall and feeds back to heater control). After the microchip mixer/reactor, the outlet of the combined initial nitration solution was combined with the initial acetyl vanillic acid solution (feeding tube 3; 4.703-4.999 mL/min) in a Vapourtec microchip mixer/reactor (1.0 mm channel width, residence volume V2=1.5 mL) at 40° C. to form the reaction mixture. The reaction mixture flowed through the microchip mixer/reactor and was collected in a vial (collector) containing deionised water to quench the nitration reaction (water:reaction mixture 2:1 v/V).

The product contained up to 67% nitro-vanillic acid (compound of formula (VI)) and as little as 22% of the side product 6-methoxy-2,4-dinitrophenol.

### Example 4—Scale Up of Nitration of Vanillic Acid in Acetic Anhydride to Form Nitro-Vanillic Acid (Compound of Formula (VI)) in a Continuous Process Using Flow Chemistry

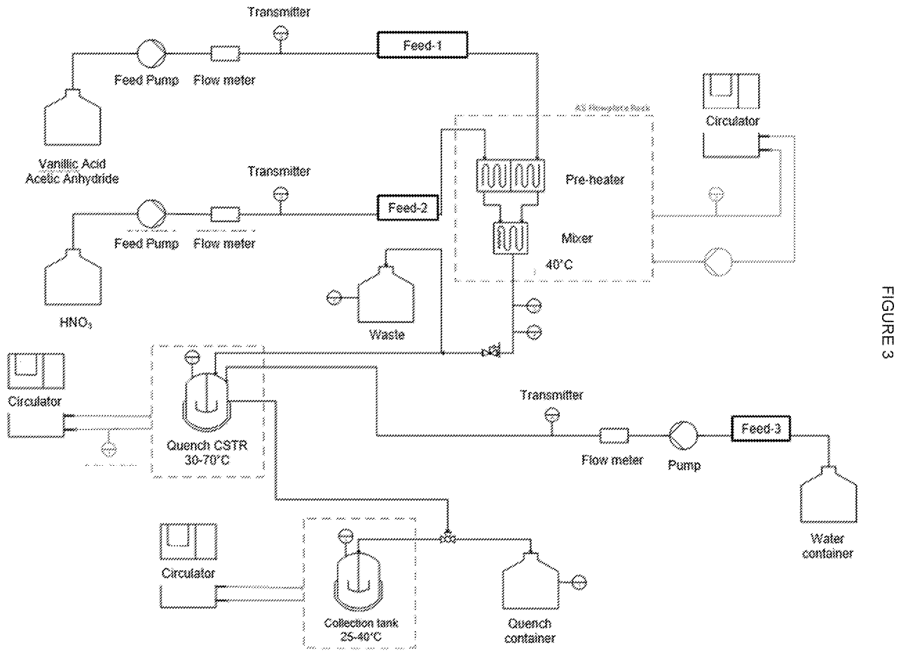

The process of Example 2 was modified so the nitric acid solution was mixed with acetyl vanillic acid solution on a larger scale. The process flow diagram is shown in FIG. 3.

Part 4a—Process Development

Generally, 10% w/w acetyl vanillic acid in acetic anhydride (based on vanillic acid starting material) was used as the initial acetyl vanillic acid solution and aqueous 65% w/w nitric acid solution was used as the initial nitration solution.

Addition of H₂SO₄ in catalytic amounts (0.2% w/w) to the 65% w/w nitric acid solution led to reaction acceleration. However, it also resulted in gas formation leading to less reliable residence time distribution in a flow system. Therefore, the inclusion of H₂SO₄ in catalytic amounts (e.g., 0.1 to 1% w/w) is preferred when the reaction rate should be maximised or when the nitration reaction occurs on smaller or batch scales. However, the inclusion of H₂SO₄ is less preferred on large scales when employing a continuous process using flow chemistry.

Increasing the concentration of acetyl vanillic acid to 15% w/w in acetic anhydride (based on vanillic acid starting material) provided a stable solution that could be stored at low temperatures (0 to 10° C., preferably 0° C.) for up to 24 hours. Therefore, these solutions are useful for storage. However, at these concentrations, precipitation/suspension was observed after addition of a nitration agent (e.g., HNO₃). Whilst this is not problematic on a small scale, it is less amenable to synthesis in a continuous process using flow chemistry. Therefore, concentrations of up to 12% w/w acetyl vanillic acid (especially up to 10% w/w) in acetic anhydride (based on vanillic acid starting material) are generally preferred because they achieve a high concentration without precipitation.

Part 4b—Sample Production and Use Test

Development was carried out using an A5-sized LL-rhombus FlowPlate® to mix the initial nitration solution and initial acetyl vanillic acid solution. This was combined with a continuous stirred tank reactor (CSTR) or semi-batch quench technology. The nitration reaction was fully mixing controlled.

Approximately 200 g of nitro-vanillic acid (compound of formula (VI)) were produced using the set up shown in FIG. 3, which utilises the CSTR quench technology, or using semi-batch quenching (not shown). The parameters employed are shown in Table 1:

| Parameters | Value |
|---|---|
| Feed-1 composition | Vanillic acid: 10.0% w/w |
| | Acetic anhydride: 89.8% w/w |
| | Others: 0.2% w/w |
| Feed-2 composition | HNO₃: 65% w/w |
| | H₂O: 35% w/w |
| Feed-3 composition | H₂O: 100% w/w |
| Flow rates | Feed-1: 109.9 g/min |
| | Feed-2: 10.1 g/min |
| | Feed-3: 120.0 g/min |
| Ratios/Stoichiometry | HNO₃/Vanillic acid: 1.6 mol/mol |
| Quench | Water/Nitration solution: |
| | 2 g/g (1.0 in CSTR, |
| | 1.0 in collection tank) |
| | Water/Nitration solution: |
| | 2 g/g (semi-batch) |
| Temperatures | Nitration: 40° C. |
| | CSTR jacket: 35° C. |
| | Collection tank: 40° C. |
| Reactor | Nitration: FlowPlate ® A5 LL-rhombus |
| | Quench: 300 mL CSTR |
| IT Guidance System | Hitec Zang Lab Vision 2.11 |

Nitro-vanillic acid (compound of formula (VI)) could be isolated almost quantitatively out of the reaction solution. Isolated material met HPLC specifications without recrystallization. Purity of the isolated material was >99 area % and assay >99 wt %. Most importantly, the yield could be increased to ~70% (68.1 to 71.7%) compared to <45% in the batch manufacturing process of WO 2013/089573.

Continuous quenching using a continuous stirred tank reactor (CSTR) gave excellent results, especially at temperatures of 40° C. and below (e.g., 4 to 40° C.). Similar results were achieved using semi-batch quenching.

The product could be used without recrystallization in the later coupling, oxidation and deprotection steps to form opicapone as described in WO 2013/089573.

### Experimental Details:

Dissolving/acetylation of vanillic acid was performed by addition of vanillic acid (86.7 g, 503 mmol) into acetic anhydride (763.3 g, 7497 mmol). Subsequently, the mixture was heated under stirring to 40° C. internal temperature until all solid was dissolved.

Nitration of acetyl vanillic acid in acetic anhydride to acetyl nitro-vanillic acid in acetic anhydride and quenching to nitro-vanillic acid in acetic acid. The flow setup consisted of a piston pump (feed-1), gear pumps (feeds-2 and -3) to introduce a solution of acetyl vanillic acid in acetic anhydride (feed-1), aqueous HNO₃ (65% w/w, feed-2) and water (feed-3). Before the experiment commenced, the reactor setup was flushed by pumping neat acetic acid at a flow rate of 20 g/min using the pump for feed-1. The line of feed-3 was flushed with water with a flow rate of 20 g/min for 5 minutes. Subsequently, aqueous HNO₃ (feed-2) was introduced into the flow system at a flow rate of 12 g/min for 5 minutes. After conditioning of feed-2 the flow rate was switched to 10.1 g/min (1.6 eq.), the acetyl vanillic acid mixture (feed-1) was introduced at a flow rate of 109.9 g/min and water was introduced at a flow rate of 120 g/min (feed-3). Feeds-1 and -2 were pre-heated in a A5 FlowPlate® (Ehrfeld, 25 mL) to 40° C. before being mixed within a microreactor (Ehrfeld FlowPlate® A5 LL-rhombus, 11 mL) at 40° C., after having passed the temperature sensor and backpressure regulator, the nitration mixture was mixed with the water in a CSTR (continuously stirred tank reactor) at 30° C. and subsequently to the collection tank at 40° C. FlowPlates®, CSTRs and collection tank were conditioned using thermostats.

To analyse the samples by HPLC a sample of the suspension after dilution with water was removed and diluted in acetonitrile (2:1 v/v).

Part 4c—Scale-Up Runs

Two scale-up runs were performed with double the flow rate of previous experiments (productivity approximately 33 kg/day).

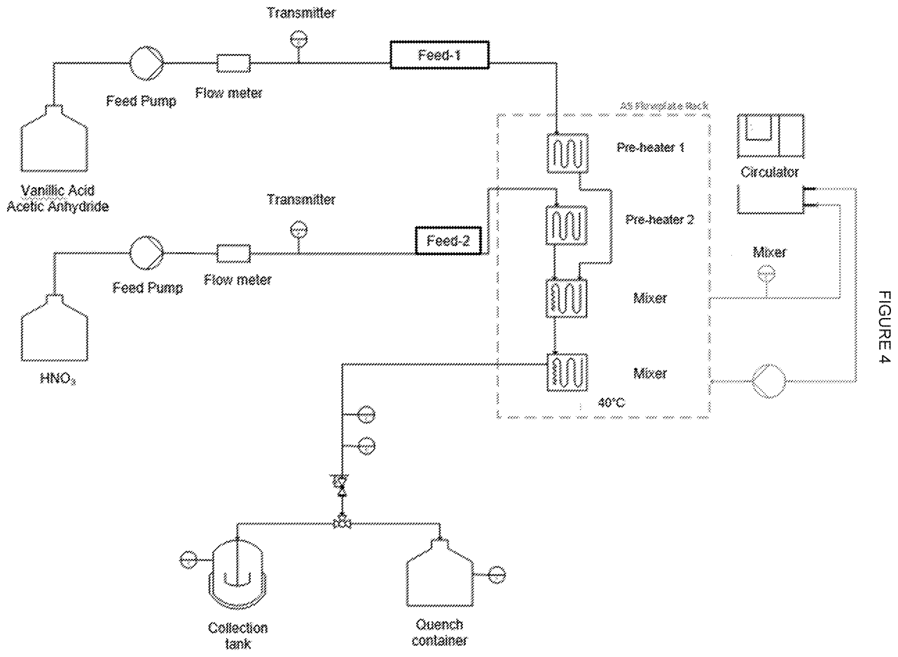

Nitro-vanillic acid (compound of formula (VI)) was produced using the set up shown in FIG. 4, which utilises batch quenching. The parameters employed are shown in Table 2:

| Parameters | Value |
|---|---|
| Feed-1 composition | Vanillic acid: 10.0% w/w |
| | Acetic Anhydride: 89.8% w/w |
| | Others: 0.2% w/w |
| Feed-2 composition | HNO₃: 65% w/w |
| | H₂0: 35% w/w |
| Flow rates | Feed-1: 201.4 g/min |
| | Feed-2: 18.6 g/min |
| Ratios/Stoichiometry | HNO₃/Vanillic acid: 1.6 mol/mol |
| Quench | Water/Nitration solution: 2 g/g |
| Temperatures | Nitration: 40° C. |
| | Collection tank: 40° C. |
| Reactor | Nitration: FlowPlates ®: A5 |
| | LL-rhombus (size 200 and 100) |
| IT Guidance System | Hitec Zang Lab Vision 2.11 |

On this larger scale, a single FlowPlate® A5 size 200 did not generate sufficient residence time for the nitration reaction to be completed. Therefore, a second FlowPlate® is preferable to maximise reaction efficiency and yield on the largest scales. This is depicted in FIG. 4. Whilst the FlowPlates® ideally have the same size (e.g., size 200), use of a second FlowPlate® of a different size (e.g., size 100) was acceptable when pressure drop (energy dissipation rate) is kept constant. The skilled person can modify the flow rates and parameters based on the teaching of this disclosure. The nitration reaction was quenched using water in a large excess after reaching steady state (e.g., 5 minutes). The steady state is achieved for example when energy dissipation rate, flow rates and conversion are kept constant, amongst other factors.

The purity of nitro-vanillic acid (compound of formula (VI)) from both runs was >99 area % and assay >99 wt. %. Yields were comparable with previous runs performed on a smaller scale (68.1 to 70.2%).

A long run experiment was also performed (5 hours) using a smaller FlowPlate® without any clogging issues. Nitration in-process controls (IPCs) were taken at 0, 2.5 and 5 hours, and were comparable to each other and previous scale-up runs, demonstrating good process robustness and scalability.

### Experimental Details:

Dissolving/acetylation of vanillic acid was performed by addition of vanillic acid (102.0 g, 593 mmol) into acetic anhydride (898.0 g, 8791 mmol). Subsequently, the mixture was heated under stirring to 40° C. internal temperature until all solid was dissolved.

Nitration of acetyl vanillic acid in acetic anhydride to acetyl nitro-vanillic acid in acetic anhydride and quenching to nitro-vanillic acid in acetic acid. The flow setup consisted of a piston pump (feed-1) and a gear pump (feed-2) to introduce a solution of acetyl vanillic acid in acetic anhydride (feed-1) and aqueous HNO₃ (65% w/w, feed-2). Before the experiment commenced, the reactor setup was flushed by pumping neat acetic acid at a flow rate of 20 g/min using the pump for feed-1. Subsequently, aqueous HNO₃ (feed-2) was introduced into the flow system at a flow rate of 12 g/min for 5 minutes. After conditioning of feed-2 the flow rate was switched to 18.6 g/min (1.6 eq.) and the acetyl vanillic acid mixture (feed-1) was introduced at a flow rate of 201.4 g/min. Feeds-1 and -2 were pre-heated in an A5 FlowPlate® (Ehrfeld, 30 mL) to 40° C. before being mixed within a microreactor (Ehrfeld FlowPlate® A5 LL-rhombus, 15.5 mL; size 200 and 21.0 mL, size 100) at 40° C. All FlowPlates® were conditioned to 40° C. using a thermostat. After the FlowPlate®, the reaction mixture was passed through a temperature sensor and a backpressure regulator set to 3 bar. The output solution was diluted/quenched with a water mixture (e.g., 2:1 w/w water mixture:output solution) resulting in the hydrolysis of the acetylated version of the compound of formula (VI) to yield the compound of formula (VI). To analyse the samples by HPLC a sample of the suspension after dilution with water was removed and diluted in acetonitrile (e.g. 2:1 v/v, acetonitrile:suspension).

### Example 5—Industrial Scale Nitration of Vanillic Acid in Acetic Anhydride to Form Nitro-Vanillic Acid (Compound of Formula (VI)) in a Continuous Process Using Flow Chemistry

The process of Example 4 is modified so the nitric acid solution is mixed with acetyl vanillic acid solution on a larger scale. Flow rates for feed-1 (10% w/w acetyl vanillic acid in acetic anhydride (based on vanillic acid starting material)) is increased to ~500 g/min; flow rates for feed-2 (65% w/w nitric acid in water) is increased to ~50 g/min; flow rates for feed-3 (water to quench) is increased to ~1000 g/min. The reaction mixture is passed through one or more (up to 6) FlowPlate® A5 (size 200/200) or a FlowPlate® A4 (size 000). It is predicted that yields of ~70% should be achieved on an industrial scale.

Other variations to the disclosed embodiments can be understood and effected by those skilled in the art in practicing the claimed invention, from a study of the drawings, the disclosure, and the appended claims. The mere fact that certain measures are recited in mutually different dependent claims does not indicate that a combination of these measures cannot be used to advantage. Any reference signs in the claims should not be construed as limiting the scope.

1. A method of preparing the compound of formula (VI): 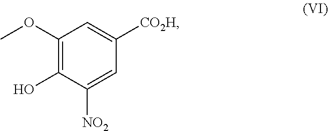
which comprises nitration of vanillic acid in an acid anhydride using a nitration agent.

2. The method of claim 1, wherein the vanillic acid is dissolved and acylated in the acid anhydride to form an initial acyl vanillic acid solution prior to nitration.

3. The method of any previous claim, wherein the acid anhydride is acetic anhydride or a halogenated derivative thereof, preferably acetic anhydride or trichloroacetic anhydride, more preferably acetic anhydride.

4. The method of claim 2, wherein the initial acyl vanillic acid solution is an initial acetyl vanillic acid solution which comprises 3.75 to 15% w/w acetyl vanillic acid in acetic anhydride.

5. The method of any previous claim, wherein the nitration agent is in the form of an initial nitration solution.

6. The method of claim 5, wherein the initial nitration solution comprises a solvent selected from the group consisting of water, H₂SO₄, acetic acid, Ac₂O, or a water/Ac₂O mixture, preferably the initial nitration solution is an aqueous solution.

7. The method of claim 5 or 6, wherein the initial nitration solution comprises 50 to 99.9% w/w nitration agent.

8. The method of any previous claim, wherein the nitration agent is HNO₃.

9. The method of any previous claim, wherein the nitration is carried out at a temperature from about 15 to about 60° C., preferably at a temperature from about 20 to about 45° C., more preferably at a temperature from about 25 to about 40° C., and optionally wherein the vanillic acid and/or acid anhydride and/or nitration agent are pre-heated before the nitration reaction.

10. The method of any previous claim, wherein the nitration reaction product is quenched with water.

11. The method of claim 10, wherein quenching is carried out from about 4 to about 40° C.

12. The method of claim 10 or 11, wherein the nitration reaction product is quenched with a 1.5- to 20-fold excess of water.

13. The method of any one of claims 1 to 12, wherein the nitration reaction is carried out as a continuous process in solution.

14. The method of claim 13, wherein the initial acyl vanillic acid solution comprises 3.75 to 15% w/w acyl vanillic acid.

15. The method of claim 13 or 14, wherein the initial nitration solution comprises 50 to 99.9% w/w nitration agent.

16. The method of any one of claims 5 to 15, wherein the initial acyl vanillic acid solution is mixed with the initial nitration solution in one or more mixer/reactors to form a reaction mixture, optionally after pre-heating the initial solutions to a temperature from about 15 to about 60° C., and wherein the nitration reaction is optionally carried out at a temperature from about 15 to about 60° C.

17. The method of claim 16, wherein the initial acyl vanillic acid solution is introduced into a first mixer/reactor at a flow rate 1- to 30-fold higher than the flow rate at which the initial nitration solution is introduced into the first mixer/reactor.

18. The method of claim 16 or 17, wherein the initial acyl vanillic acid solution is introduced into a first mixer/reactor at a flow rate of 0.1 to 600 g/minute.

19. The method of any one of claims 16 to 18, wherein the initial nitration solution is introduced into a first mixer/reactor at a flow rate of 0.1 to 60 g/minute.

20. The method of any one of claims 16 to 19, wherein the reaction mixture flows to a second mixer/reactor after mixing in the first mixer/reactor, wherein the first and/or second mixer/reactors are optionally heated to a temperature from about 15 to about 60° C., and wherein the nitration reaction is optionally carried out at a temperature from about 15 to about 60° C.

21. The method of any one of claims 16 to 20, wherein the reaction mixture subsequently flows to a collector.

22. The method of claim 21, wherein the reaction mixture is quenched with water in the collector to form a quenched reaction mixture.

23. The method of claim 22, wherein the water is introduced into the collector at a flow rate 1- to 2-fold higher than the flow rate at which the reaction mixture is introduced into the collector.

24. The method of claim 22 or 23, wherein the water is introduced into the collector at a flow rate of 50 to 1500 g/minute.

25. The method of any one of claims 22 to 24, wherein the quenched reaction mixture flows to a precipitator.

26. The method of any previous claim, wherein the compound of formula (VI) is produced from vanillic acid in an overall yield of 50% or greater.

27. The method of any previous claim, wherein the compound of formula (VI) is precipitated, filtered and dried.

28. The method of claim 27, wherein the compound of formula (VI) is recovered in a crystalline form without an additional recrystallization step.

29. A compound of formula (VI): 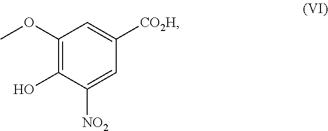
produced by the process of any one of claims 1 to 28.

30. A method according to any one of claims 1 to 28, wherein a compound of formula (V) 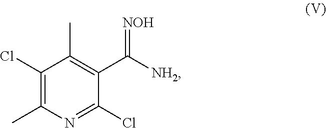
is reacted with the compound of formula (VI) to form a compound of formula (III) 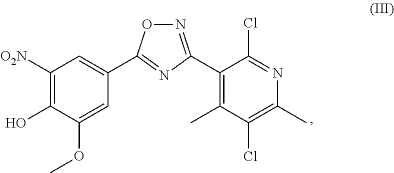 [SMILES: COc1cc(-c2nc(-c3c(Cl)nc(C)c(Cl)c3C)no2)cc([N+](=O)[O-])c1O ; ","]
wherein the compound of formula (VI) does not undergo an additional recrystallization step.

31. A compound of formula (III) 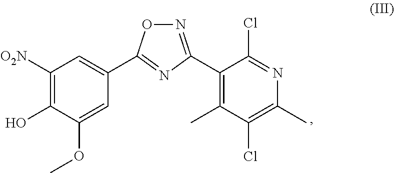 [SMILES: COc1cc(-c2nc(-c3c(Cl)nc(C)c(Cl)c3C)no2)cc([N+](=O)[O-])c1O ; ","]
produced by the process of claim 30.

32. A method according to claim 30, wherein the compound of formula (III) is oxidised to form a compound of formula (I):

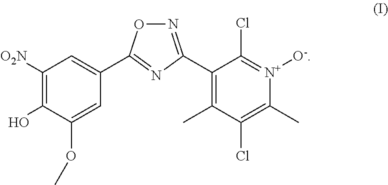

33. A compound of formula (I): 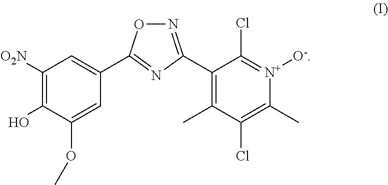
produced by the process of claim 32.

34. A method according to claim 32, wherein the compound of formula (I) undergoes O-demethylation and optional pharmaceutically acceptable salt formation to form a compound of formula (II) or a pharmaceutically acceptable salt thereof:

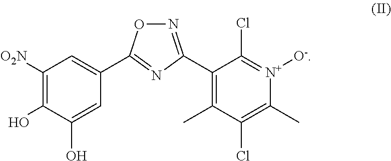

35. A compound of formula (II) or a pharmaceutically acceptable salt thereof: 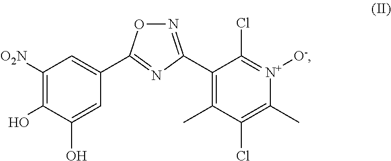
produced by the process of claim 34.

36. A solution of an acyl nitro-vanillic acid in an acid anhydride, preferably acetyl nitro-vanillic acid in acetic anhydride.

37. The solution of claim 36, wherein the solution comprises 4.6 to 18.2% w/w of the acetyl nitro-vanillic acid at room temperature and pressure, preferably 7.6 to 15.2% w/w.

38. A solution of an acyl vanillic acid in an acid anhydride, preferably acetyl vanillic acid in acetic anhydride.

39. The solution of claim 38, wherein the solution comprises 3.75 to 18.75% w/w of the acetyl vanillic acid at room temperature and pressure, preferably 3.75 to 15% w/w, more preferably 6.25 to 12.5% w/w.
